(19) United States
(12) Patent Application Publication
(10) Pub. No.: US 2007/0181134 A1
(43) Pub. Date: Aug. 9, 2007

(54) Methods for Making Male Condom of Self-standing Skirt Structure etc

(76) Inventor: Bo LANG, Shenzhen (CN)

Correspondence Address: BO LANG, 1401 HUATONG BUILDING, SUNGAG EAST ROAD, SHENZHEN, GD 518008, CN

(21) Appl. No.: 11/277,235

(22) Filed: Mar. 23, 2006

(30) Foreign Application Priority Data
Feb. 7, 2006 (CN) 2006 102000980
Mar. 6, 2006 (CN) 2006 102002079

(51) Int. Cl. A61F 6/04

(52) U.S. Cl. 128/844

(57) ABSTRACT

A method for making male condom of self-standing skirt structure, a skirt-supporting thickened belt-like ring coming together with said skirt structure and located at the root area of said condom, a rim at the middle line of the condom body, comprising the steps of: adding a taper shape structure onto the bottom of conventional condom mold; covering said condom mold with a mold cover being split into several parts, with said taper shape structure, the root area of said condom mold and the middle line of said condom mold are being exposed and uncovered; using nozzles to evenly spray coagulant onto the parts of the mold exposed; opening said mold cover; drying said mold in a drying oven; dipping said condom mold into a dipping trough and then following conventional condom dipping workmanship.

### FIELD OF THE INVENTION

The present invention relates to a male condom, and more specifically, methods for making male condom of a self-standing skirt structure, a skirt-supporting thickened belt-like ring coming together with said skirt structure and located at the root area of the condom, a rim at the middle line of the condom body serving as the starting axis to roll up the condom.

### BACKGROUND ART

Body liquid is one of the channels to spread AIDS virus. Condoms have long been adopted to prevent AIDS besides of its contraceptive function. But conventional male condom can only cover the shaft of penis while the root and pubis area are exposed and uncovered. This causes the root and pubis area to be contaminated by body liquid from female vulva, or vise versa, female vulva being contaminated by the root and pubis area of a male. Although theoretically AIDS virus can not survive in open air for hours, in practice it's not reasonable to assume people should stand naked without their pants on for hours in order for the air to kill AIDS virus. There are numerous cases proved that people got AIDS even they had worn conventional male condom during sex. Female condom has an extended part in its open end. But it has to be inserted into female vagina before use therefore female dislikes it because it feels like an alien object inside, male dislikes it because they feel they are inserting into a plastic bag. In most of the cases, sex is driven by the male part. To better prevent AIDS etc Sex-Transmitted-Diseases, a male condom can cover the root and pubis area is needed.

There are male condom for medical purpose and fancy sex toy in condom shape and both of them are made from latex in most of the cases. The male condom for medical purpose usually is made with latex dipping workmanship and it's thin and soft enough to be rolled up before use. The sex toy in condom shape usually is made with latex injection workmanship and it's thick and hard therefore can not be rolled up.

There are attempts to made a male condom for medical purpose with a big skirt, but combination of two different workmanships, i.e., the skirt is made with injection or extrusion workmanship therefore it's rigid while the condom body is made with dipping workmanship therefore it's soft, will make it difficult to put the two pieces together and sticking or pasting will lead to leakage at where the two pieces join together, or otherwise, it's in low efficiency to work out such a condom.

There are also attempts to make a male condom for medical purpose with a big skirt by conventional dipping workmanship. But multiple dipping workmanship will make both the skirt and the condom body thick and rigid therefore loses the sexual sensitivity that people need. Or otherwise, one time dipping will make both the skirt and the condom body thin and soft therefore the skirt can not stand up to isolate body liquid contamination. That's to say, conventional dipping workmanship is good to produce condom parts of the same thickness. It has never been used to produce condom parts of different thickness in same one action.

There are inventions to put adhesives on the back of the soft skirt structure trying to hold it there, but as love-making is a violent exercise, if the adhesives are strong enough to hold the soft skirt there, it can also peel off some skin when being taken off; if the adhesives are not strong enough to hold the soft skirt there, it has no reason to be used in the first place.

Progress on conventional male condom has to be tiny in its each step otherwise people won't accept it. Therefore modifications of practical value might become not so obvious sometimes. Before this invention, there's no matured relative product in market place.

### SUMMARY OF THE INVENTION

It is therefore an object of this invention to provide a male condom of a self-standing skirt structure to isolate body liquid contamination. The condom body has to reserve its softness & thinness to keep the desirable sexual sensitivity, in the meantime the skirt structure has to be rigid and self-standing in order to function as it promised.

It is another object of this invention to provide simple methods and relative equipment including mold to use conventional latex dipping workmanship and the producing line to produce a condom of different thickness and hardness in its different parts in same one action.

It is yet another object of this invention to keep the self-standing skirt in place and won't flip over during usage.

It is a further object of this invention to provide a condom that can enhance the erection of penis so that it can bring more joy to people.

As this condom become more complicated in shape, it is another object of this invention to provide a solution on how to roll up the condom so that people can use it in a way that they are familiar with.

The present invention in one embodiment provides a method for making male condom of self-standing skirt structure, a skirt-supporting thickened belt-like ring coming together with said skirt structure and located at the root area of said condom, a rim at the middle line of the condom body, comprising the steps of adding a taper shape structure onto the bottom of conventional condom mold, covering said condom mold with a mold cover being split into several parts, with contacting edges of said parts horizontally hugged in concavo-convex shape, while said taper shape structure, the root area of said condom mold and the middle line of said condom mold are being exposed and uncovered, using nozzles to evenly spray coagulant onto said taper shape structure, the root area of said condom mold and the middle line of said condom mold, opening said mold cover, drying said mold in a drying oven, dipping said condom mold into a dipping trough and then following conventional condom dipping workmanship.

According to this invention, coagulant will have chemical reactions with liquefied latex. The latex touching the parts of the condom mold coated with coagulant will become much thicker and harder around 0.2 mm in thickness. While without coagulant, normal dipping workmanship will make the latex thin and soft around 0.03 mm in thickness. The taper shape structure, the root area of said condom mold and the middle line of said condom mold are being coated with coagulant in this invention, therefore latex on these parts will become much thicker and harder around 0.2 mm in thickness after dipping. Those parts being covered and untouched with coagulant, specifically the mold parts for condom body, will make latex in conventional thinness and softness with a thickness around 0.03 mm.

According to this invention, the skirt structure, the skirt-supporting belt-like ring coming together with said skirt structure and located at the root area of said condom, the rim at the middle line of the condom body are much thicker and harder with a thickness around 0.2 mm while the condom body reserves its thinness & softness with a thickness around 0.03 mm. As the skirt structure is thicker and harder, it won't become soft & weak therefore it can stand by itself. But the skirt structure could flip over toward female side during usage, therefore a supporting structure to keep it unilateral directional toward male side is needed. The belt-like ring coming together with said skirt structure and located at the root area of said condom is thicker and harder and can prevent the skirt structure from flipping over. The rim at the middle line of the condom body is thick and rigid enough to serve as the starting axis to roll up the condom when the condom body is folded in half. Thus a male condom of self-standing skirt structure, a skirt-supporting thickened belt-like ring coming together with said skirt structure and located at the root area of said condom, a rim at the middle line of the condom body, in the meantime the condom body of conventional thinness and softness, is accomplished.

### BRIEF DESCRIPTION OF THE DRAWINGS

Various other objects, advantages, and features of the invention will become apparent to those skilled in the art from the following discussion taken in conjunction with the following drawings, in which:

FIG. 1 illustrates the equipment including condom cover in its opening and stand-by position.

FIG. 2 illustrates the equipment including condom cover in its working status.

FIG. 3 illustrates the top view of the cross-section of the condom cover.

FIG. 4 illustrates the side view of the location of the rim when said male condom being folded in half.

FIG. 5 illustrates said male condom after being rolled up.

FIG. 6 illustrates an alternate form of the condom mold with a column shape structure perpendicular to the side of the condom mold.

FIG. 7 illustrates said male condom being made according to this invention.

FIG. 8 illustrates said male condom of an elastic loop on the skirt edge.

FIG. 9 illustrates said male condom of screw texture on the condom body.

FIG. 10 illustrates said male condom of dots on the condom body.

FIG. 11 illustrates said male condom of sunken shapes on the condom body and spine shapes on the head of the condom body.

### DETAILED DESCRIPTION OF THE INVENTION

Detailed embodiments of the present invention are disclosed herein with reference to the drawings.

FIG. 1 to FIG. 7 show the preferred embodiment of the present invention.

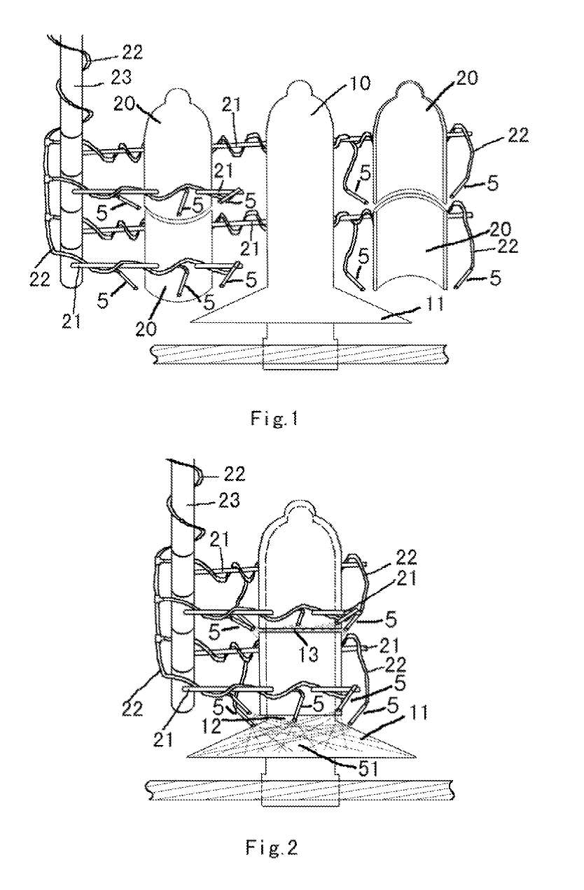

Referring to FIG. 1 and FIG. 2, they illustrate a method for making male condom 30 of self-standing skirt structure 33, a skirt-supporting thickened belt-like ring 34 coming together with said skirt structure and located at the root area of said condom, a rim 35 at the middle line of the condom body, comprising the steps of: adding a taper shape structure 11 onto the bottom of conventional condom mold 10; covering said condom mold with a mold cover 20 being split into several parts, while said taper shape structure 11, the root area 12 of said condom mold and the middle line 13 of said condom mold are being exposed and uncovered; using nozzles 5 to evenly spray coagulant 51 onto said taper shape structure 11, the root area 12 of said condom mold and the middle line 13 of said condom mold; opening said mold cover 20; drying said mold in a drying oven; dipping said condom mold into a dipping trough and then following conventional condom dipping workmanship. In this way, said self-standing skirt structure 33, said condom body 32 and the sperm reservoir 31, said skirt-supporting thickened belt-like ring 34 coming together with said skirt structure and located at the root area of said condom 30, said rim 35 at the middle line of the condom body 32 are being produced by same one dipping workmanship therefore seamless.

An alternative method for making male condom 30 of self-standing skirt structure 33, a skirt-supporting thickened belt-like ring 34 coming together with said skirt structure and located at the root area of said condom, a rim 35 at the middle line of the condom body, comprising the steps of: adding a taper shape structure 11 onto the bottom of conventional condom mold 10; lowering down a mold cover 20 being split into upper and lower two parts to cover said condom mold, with said taper shape structure 11, the root area 12 of said condom mold and the middle line 13 of said condom mold are being exposed and uncovered; rotating said mold cover 20 around said mold; using nozzles 5 on said rotating mold cover 20 to evenly spray coagulant 51 onto said taper shape structure 11, the root area 12 of said condom mold and the middle line 13 of said condom mold; raising up and taking back said mold cover 20; drying said mold in a drying oven; dipping said condom mold into a dipping trough and then following conventional condom dipping workmanship.

FIG. 1 and FIG. 2 also show an automated auxiliary equipment being added onto conventional latex dipping producing line to treat condom molds in lots before it can produce a condom of different thickness and hardness in its different parts in same one action, comprising: a condom mold cover 20 being split into several parts in a similar shape of condom mold but larger than condom mold to cover said condom mold; mechanical arms 21 to move the condom mold cover 20 parts to cover or uncover said condom mold; spray nozzles 5; pipes 22 to connect said nozzles 5 with storage tank of coagulant 51; metal structure 23 to hang equipment parts around condom mold; gearing to convey condom molds onto and away from treatment assembly; motor(s) to drive said assembly; pump to transfer coagulant 51 from the storage tank to spray nozzles 5; sensors to position the location of said condom molds; PLC module or micro-processor to control said assembly; elastic supporter in both sides of said assembly to balance and amortize condom molds groups.

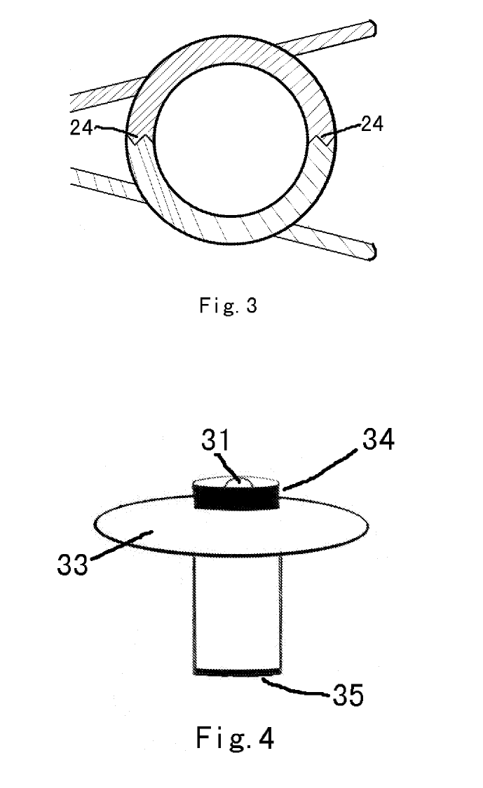

FIG. 3 illustrates the contacting edges of said condom cover 20 that are in concavo-convex shape 24 when hugging horizontally to prevent the coagulant 51 from saturating into the covered parts of the mold for condom body.

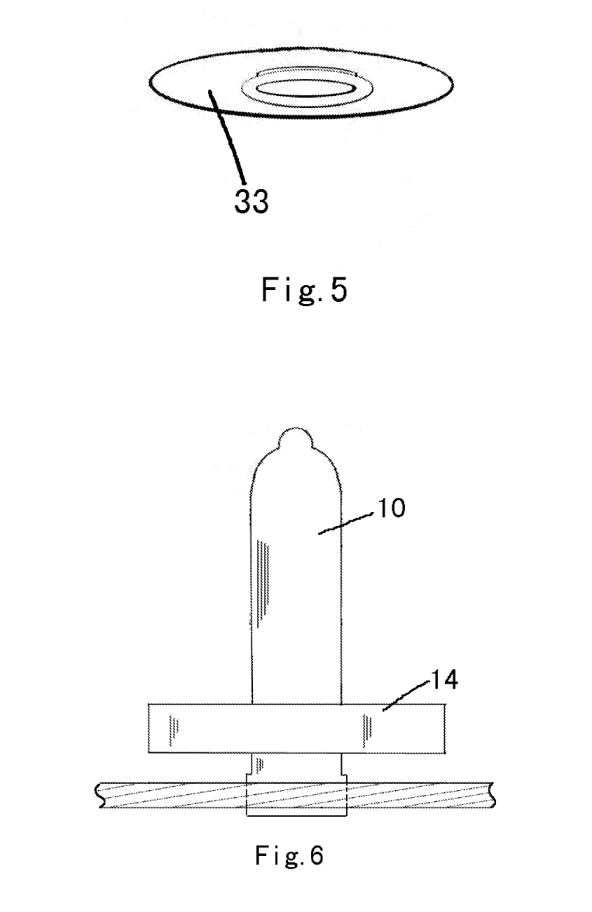

FIG. 4 and FIG. 5 show the method to roll up and pack said male condom, comprising the steps of: raising up said skirt structure 33 till said condom being folded in half with the sperm reservoir 31 of said condom standing upwardly; blowing compressed air downward between the two layers of the folded condom till the rim 35 at the middle line of the condom body is level off; grasping the rim 35 now on the bottom of folded condom; putting said condom onto a rod of the condom rolling equipment; coiling said condom with the rim 35 serving as the starting axis till the roll reaching the bottom of the self-standing skirt structure 33; packing the rolled up condom in a conventional means.

FIG. 6 shows an alternate form of mold 10 for making said male condom, the taper shape structure 11 turns into a column shape structure 14 when the upper surface of said taper shape structure 11 becomes perpendicular to the sides of said condom mold 10. Materials to make said mold are including but not limited to: steel, aluminum etc metal materials or glass, porcelain, rubber, high-molecule plastic etc nonmetal materials.

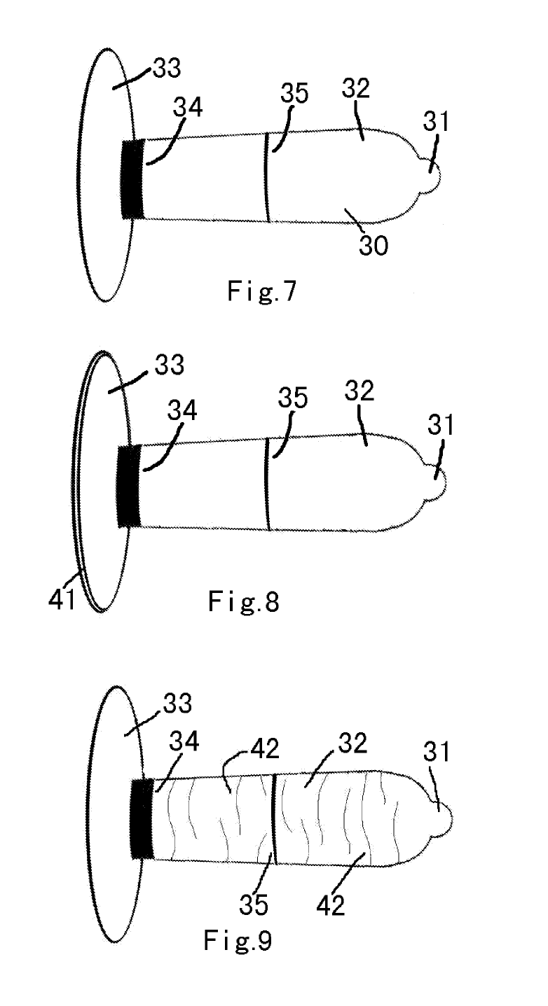

FIG. 7 illustrates the male condom 30 comprising a sperm reservoir 31 at the closing end of the condom; a tubular condom body 32; wherein said condom body 32 has a harder and thicker self-standing skirt structure 33 at the open end; a skirt-supporting thickened belt-like ring 34 coming together with said skirt structure and located at the root area of said condom body; a rim 35 at the middle line of said condom body serving as the starting axis to roll up the condom when said condom body being folded in half.

According to this invention, coagulant 51 will have chemical reactions with liquefied latex. The latex touching the parts of the condom mold coated with coagulant 51 will become much thicker and harder around 0.2 mm in thickness. While without coagulant 51, normal dipping workmanship will make the latex thin and soft around 0.03 mm in thickness. The taper shape structure 11, the root area 12 of said condom mold and the middle line 13 of said condom mold are being coated with coagulant 51 in this invention, therefore latex on these parts will become much thicker and harder around 0.2 mm in thickness after dipping. Those parts being covered and untouched with coagulant, specifically the mold parts for condom body, will make latex in conventional thinness and softness with a thickness around 0.03 mm.

According to this invention, the skirt structure 33, the skirt-supporting belt-like ring 34 coming together with said skirt structure 33 and located at the root area of said condom, the rim 35 at the middle line of the condom body 32 are much thicker and harder with a thickness around 0.2 mm while the condom body 32 reserves its thinness & softness with a thickness around 0.03 mm. In a thickness around 0.2 mm, the skirt structure 33 is rigid enough that after being bended, it can bounce back and resume its self-standing shape. As the skirt structure 33 is thicker and harder, it won't become soft & weak therefore it can stand by itself. But the skirt structure 33 could flip over toward female side during usage, therefore a supporting structure to keep it unilateral directional toward male side is needed. The belt-like ring 34 coming together with said skirt structure 33 and located at the root area of said condom 30 is thicker and harder and can prevent the skirt structure 33 from flipping over. The rim 35 at the middle line of the condom body 32 is thick and hard enough to serve as the starting axis to roll up the condom 30 when the condom body 32 is folded in half. Thus a male condom 30 of self-standing skirt structure 33, a skirt-supporting thickened belt-like ring 34 coming together with said skirt structure 33 and located at the root area of said condom, a rim 35 at the middle line of the condom body 32, in the meantime the condom body of conventional thinness and softness, is accomplished.

Another function of the skirt-supporting thickened belt-like ring 34 coming together with said skirt structure 35 and located at the root area of said condom is that it's equivalent to the rim of the eye of a lamb that being adopted since ancient China. The ring 34 can reduce the speed of the flow back of the blood in the vein of the penis but it does not holdback the pump in of the blood in the artery. Therefore it will increase the amount of the blood inside the sponge-like muscle of the penis. Just like doing gym often can enlarge muscles, the frequent congestion of the sponge-like muscle of the penis will lengthen and increase the fiber of the muscle of the penis. Therefore frequent adoption of this invention will help the male to enlarge penis, treat impotence and enjoy a longer sex time.

The materials to make said male condom in present invention are including but not limited to: rubber, latex, plastic, polyurethane, paper etc. Said male condom can be made non-transparent, semi-transparent or completely transparent. By adding coloring matters of different kinds into the raw materials, said male condom can be in different fancy color(s). When partially or the whole said condom is coated with fluorescent material, it can give out dim light in darkness or night time.

The male condom in present invention usually is lubricated before use. When replacing the lubricants with fruit flavor etc essence, it can be used especially as oral-sex condom.

When adding spermicide into the male condom 30 in present invention, it has better effect for contraceptive purpose.

When adding narcotic into the male condom 30 in present invention, it can make the glans anaesthesia so that the male can endure a longer sex time.

FIG. 8 shows the second embodiment of the present invention, wherein the condom body 32 additionally comprising a big elastic plastic loop 41 at the outer edge of said skirt structure 33 to stretch out said skirt structure to make it self-stand.

The method for making such a male condom comprising the steps of: making said condom body 32 together with the skirt structure 33 using conventional one time latex dipping workmanship or polyurethane forming workmanship; melting or sticking a big elastic plastic loop 41 onto the outer edge of said skirt structure 33 to stretch out said skirt structure 33 to make it self-stand; melting or sticking a thickened belt-like ring 34 onto the root area of said condom; melting or sticking a rubber rim 35 at the middle line of the condom body.

FIG. 9 shows the third embodiment of the present invention, wherein the condom body 32 additionally comprising screw thread texture 42 on the outer surface of said condom body 32.

Another alternative method for making male condom of self-standing skirt structure 33, a skirt-supporting thickened belt-like ring 34 coming together with said skirt structure 34 and located at the root area of said condom, a rim 35 at the middle line of the condom body 32, comprising the steps of: making the thin and soft condom body using conventional one time dipping workmanship using a mold of protruding screw thread texture; making the thicker and harder self-standing skirt structure 33 together with said thickened belt-like ring 34 at the root area of said condom using twice or multiple dipping workmanship or injection or extruding etc workmanships; connecting the thin and soft condom body 32 and the thicker and harder self-standing skirt structure 31 together with said thickened belt-like ring 34 at the root area of said condom using heat-melting, ultrasonic-melting, high frequency melting or glue sticking etc methods; melting or sticking a rubber rim 35 at the middle line of the condom body 32.

According to principles in human body engineering science and bionics, the continuous or broken screw thread texture 42 on the outer surface of said condom body 32 will simulate the status of prepuce to give female more realistic feeling.

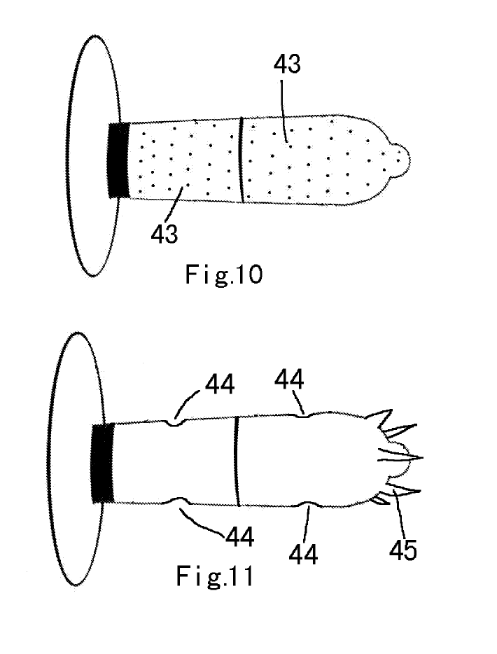

FIG. 10 shows the fourth embodiment of the present invention, wherein the condom body 32 additionally comprising dots 43 on the outer surface of said condom body.

According to principles in human body engineering science and bionics, the raised dots 43 on the outer surface of said condom body 32 will bring more stimulation to female.

FIG. 11 shows the fifth embodiment of the present invention, wherein the condom body 32 additionally comprising: sunken shape(s) 44 on the root, middle or the head area of said condom body 32 and spine shapes 45 on the head of said condom body 32.

Sunken shapes 44 on the root or middle area of said condom body 32 will give different pressure on the shaft of penis. Sunken shape 44 near the head area of said condom body 32 will make the condom body 32 to fit better with the glans. Spine shapes 45 on the head of said condom body 32 will give more stimulation to female.

Although the invention has been described by reference to particular illustrative embodiments thereof, many changes and modifications of the invention may become apparent to those skilled in the art without departing from the spirit and scope of the invention. It is therefore intended to include within this patent all such changes and modifications as may reasonably and properly be included within the scope of the present invention's contribution to the art.

What is claimed is:

1. A method for making male condom of self-standing skirt structure, a skirt-supporting thickened belt-like ring coming together with said skirt structure and located at the root area of said condom, a rim at the middle line of the condom body, comprising the steps of:
adding a taper shape structure onto the bottom of conventional condom mold;
covering said condom mold with a mold cover being split into several parts, with said taper shape structure, the root area of said condom mold and the middle line of said condom mold are being exposed and uncovered;
using nozzles to evenly spray coagulant onto said taper shape structure, the root area of said condom mold and the middle line of said condom mold;
opening said mold cover;
drying said mold in a drying oven;
dipping said condom mold into a dipping trough and then following conventional condom dipping workmanship.

2. An alternative method for making male condom of self-standing skirt structure, a skirt-supporting thickened belt-like ring coming together with said skirt structure and located at the root area of said condom, a rim at the middle line of the condom body, comprising the steps of:
adding a taper shape structure onto the bottom of conventional condom mold;
lowering down a mold cover being split into upper and lower two parts to cover said condom mold, with said taper shape structure, the root area of said condom mold and the middle line of said condom mold are being exposed and uncovered;
rotating said mold cover around said mold;
using nozzles on said rotating mold cover to evenly spray coagulant onto said taper shape structure, the root area of said condom mold and the middle line of said condom mold;
raising up and taking back said mold cover;
drying said mold in a drying oven;
dipping said condom mold into a dipping trough and then following conventional condom dipping workmanship.

3. Another alternative method for making male condom of self-standing skirt structure, a skirt-supporting thickened belt-like ring coming together with said skirt structure and located at the root area of said condom, a rim at the middle line of the condom body, comprising the steps of:
making the thin and soft condom body using conventional one time dipping workmanship;
making the thicker and harder self-standing skirt structure together with said thickened belt-like ring at the root area of said condom using twice or multiple dipping workmanship or injection or extruding etc workmanship;
connecting the thin and soft condom body and the thicker and harder self-standing skirt structure together with said thickened belt-like ring at the root area of said condom using heat-melting, ultrasonic-melting, high frequency melting or glue sticking etc methods;
melting or sticking a rubber rim at the middle line of the condom body.

4. A further alternative method for making male condom of self-standing skirt structure, a skirt-supporting thickened belt-like ring coming together with said skirt structure and located at the root area of said condom, a rim at the middle line of the condom body, comprising the steps of:
making said condom body together with the skirt structure using conventional one time latex dipping workmanship or polyurethane forming workmanship;
melting or sticking a big elastic plastic loop onto the outer edge of said skirt structure to stretch out said skirt structure to make it self-stand;
melting or sticking a thickened belt-like ring onto the root area of said condom;
melting or sticking a rubber rim at the middle line of the condom body.

5. A male condom, comprising:
a sperm reservoir at the closing end of the condom;
a tubular condom body;
wherein said condom body is of:
a rigid and thicker self-standing skirt structure of the same material of the condom body at the open end of the condom body and being seamlessly connected with the condom body via a belt-like ring;
a skirt-supporting thickened belt-like ring coming together with said skirt structure and located at the root area of said condom body;
a rim at the middle line of said condom body serving as the starting axis to roll up the condom when said condom body being folded in half;
in the meantime the tubular condom body reserves its conventional thinness and softness.

6. The male condom of claim 5, additionally comprising a big elastic plastic loop at the outer edge of said skirt structure to stretch out said skirt structure to make it self-stand.

7. The male condom of claim 5, additionally comprising:
screw thread texture on the outer surface of said condom body;
dots on the outer surface of said condom body;
sunken shape(s) on the root, middle or the head area of said condom body;
spine shapes on the head of said condom body.

8. The male condom of claim 5, wherein said condom is added with fruit flavor etc essence, or lubricants, spermicide, narcotic etc chemicals.

9. An automated auxiliary equipment being added onto conventional latex dipping producing line to treat condom molds in lots before it can produce a condom of different thickness and hardness in its different parts in same one action, comprising:
a condom mold cover being split into several parts in a similar shape of condom mold but larger than condom mold to cover said condom mold;
mechanical arms to move the condom mold cover parts to cover or uncover said condom mold;
spray nozzles;
pipes to connect said nozzles with storage tank of coagulant;
metal structure to hang equipment parts around condom mold;
gearing to convey condom molds onto and away from treatment assembly;
motor(s) to drive the assembly;
pump to transfer coagulant from the storage tank to spray nozzles;
sensors to position the location of said condom molds;
PLC module or micro-processor to control said assembly;
elastic supporter in both sides of said assembly to balance and amortize condom molds groups.

10. The automated auxiliary equipment of claim 9, wherein the contacting edges of said condom cover are in concavo-convex shape when hugging horizontally.

11. A mold for making said male condom, comprising an additional taper shape structure on the bottom position of said mold, wherein an alternate form of said taper shape structure turns into column shape structure when the upper surface of said taper shape structure becomes perpendicular to the sides of said condom mold.

12. A method for rolling up and pack said male condom, comprising the steps of:
raising up said skirt structure till said male condom being folded in half with the sperm reservoir of said condom standing upwardly;
blowing compressed air downward between the two layers of the folded condom till the rim at the middle line of the condom body level off, grasping the rim now on the bottom of folded condom;
putting said condom onto a rod of the condom rolling equipment;
coiling said condom with the rim serving as the starting axis till the roll reaching the bottom of the self-standing skirt structure;
packing the rolled up condom in a conventional means.
